(19) United States
(12) Patent Application Publication
(10) Pub. No.: US 2025/0195828 A1
(43) Pub. Date: Jun. 19, 2025

(54) URINARY BAG REDUCING URINE BLOCKAGE

(71) Applicant: CHIEN-CHUNG SU, Taichung City (TW)

(72) Inventors: CHIEN-CHUNG SU, Taichung City (TW); YI-CHIA CHANG, Taichung City (TW); YA-WEN KU, Taichung City (TW); PING-YI HSU, Taichung City (TW)

(21) Appl. No.: 18/979,712

(22) Filed: Dec. 13, 2024

(30) Foreign Application Priority Data
Dec. 19, 2023 (TW) 112149599

(51) Int. Cl. A61M 25/00

(52) U.S. Cl. CPC A61M 25/0017; A61M 25/002; A61M 2202/0496

(57) ABSTRACT

An urinary bag reducing urine blockage includes an urinary bag body having a through bore, a catheter, and a bend preventing tube. The catheter has one end connected with an urethra of a user, and the other end passing through the through bore of the urinary bag body. The bend preventing tube has a flow bore therein, and includes an insertion part and a supporting part. The catheter is in communication with the flow bore and mounted around the insertion part. The insertion part, keeps the catheter to be straight. The supporting part is located inside the urinary bag body. The outer diameter of the supporting part is larger than the outer diameter of the insertion part. The supporting part supports the interior of the urinary bag body. Thus, the bend preventing tube prevents the catheter and the urinary bag body from bending to cause urine blockage, ensuring smooth urine drainage.

### BACKGROUND OF THE INVENTION

### 1. Field of the Invention

The present invention relates to urinary bag structures, and more particularly, to an urinary bag reducing urine blockage.

### 2. Description of the Related Art

Patients are unable to urinate voluntarily (such as those undergoing or underwent surgery) or individuals with disabilities usually require the use of an urinary bag. Referring to FIG. 7 and FIG. 8, a conventional urinary bag structure comprises an urinary bag body 91, a catheter 92, and a hanger 93. The two upper corners of the urinary bag body 91 are provided with two fixing bores 911. The catheter 92 has a first connection part 921 and a second connection part 922. The first connection part 921 is connected to the urethra of the user, while the second connection part 922 passes through the urinary bag body 91 for facilitating the communication therewith. A through hole 931 is provided at the lower middle position of the hanger 93, with two hooking parts 932 extending downward and two hooks 933 extending upward on both sides of the hanger 93.

During use, the catheter 92 passes through the through hole 931 of the hanger 93, and the two hooking parts 932 of the hanger 93 are hooked onto the two fixing bores 911 of the urinary bag body 91. The two hooks 933 of the hanger 93 are hooked onto the bedside or other suitable locations, thereby allowing the urine of the user to flow into the urinary bag body 91 via the catheter 92 for urine collection.

However, in conventional urinary bag structures, the urinary bag body 91 often deforms due to the weight of the collected urine, as shown in FIG. 9. This deformation causes the catheter 92 to be bent in adjacent to the urinary bag body 91, such that the second connection part 922 of the catheter 92 is pressed against the inner wall of the urinary bag body 91, resulting in the urine blockage issue hindering the normal urinary drainage process. Also, due to the structural complexity, the assembly of the conventional urinary bag is inconvenient, and the manufacturing cost thereof is relatively high, failing to meet the economic efficiency.

### SUMMARY OF THE INVENTION

In view of the aforementioned issues of conventional urinary bag structures, the inventor of the present invention invented the idea through extensive discussions, sample testing, and multiple iterations of revisions and improvements, whereby the present invention is accordingly introduced.

An embodiment of the present invention provides an urinary bag reducing urine blockage, comprising:
an urinary bag body having a through bore on an upper side thereof, the through bore in communication with an interior of the urinary bag body;
a catheter connected and in communication with the urinary bag body, the catheter having a first connection part and an opposite second connection part, the first connection part used to be connected with an urethra of a user, the second connection part inserted into the urinary bag body from the through bore and placed inside the urinary bag body; and
a bend preventing tube connected and in communication between the urinary bag body and the catheter, the bend preventing tube having an insertion part and a supporting part, with a flow bore passing through the insertion part and the supporting part, the second connection part of the catheter mounted around the insertion part, the flow bore in communication with the catheter, the insertion part keeping the catheter to be straight; the supporting part located inside the urinary bag body, an outer diameter of the supporting part being larger than an outer diameter of the insertion part, the supporting part supporting the interior of the urinary bag body.

With such configuration, the urinary bag reducing urine blockage disclosed by the present invention comprises the bend preventing tube disposed between the catheter and the urinary bag body, wherein the insertion part of the bend preventing tube keeps the catheter to be straight, and the supporting part of the catheter supports the urinary bag body. Therefore, the urine blockage issues is prevented, ensuring the normal operation of urine drainage, thereby resolving the issues of the conventional urinary bag being easily bent and suffering from urine blockage.

Also, the present invention is structurally simple, facilitating a quick assembly of the urinary bag, and lowering the overall manufacturing cost, meeting the economic efficiency.

### BRIEF DESCRIPTION OF THE DRAWINGS

FIG. 1 is a perspective view of the present invention.

FIG. 2 is an exploded view of the present invention.

FIG. 3 is a sectional view of the present invention.

FIG. 4 is an enlarged view of part A1 in FIG. 3.

FIG. 5 is a schematic view illustrating the operation status of the present invention.

FIG. 6 is an enlarged view of part A2 in FIG. 5.

FIG. 7 is a perspective view of a conventional urinary bag.

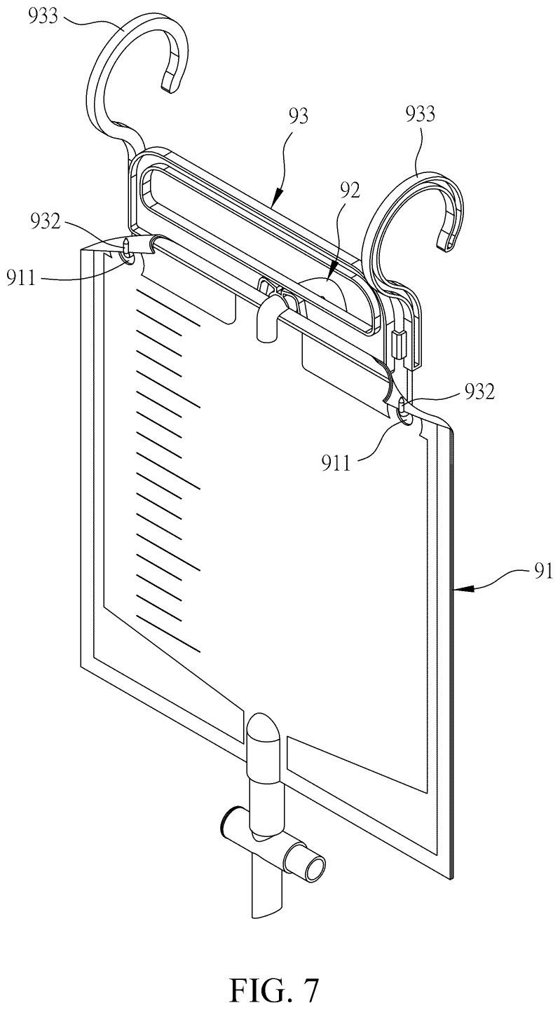

FIG. 8 is a sectional view of a conventional urinary bag.

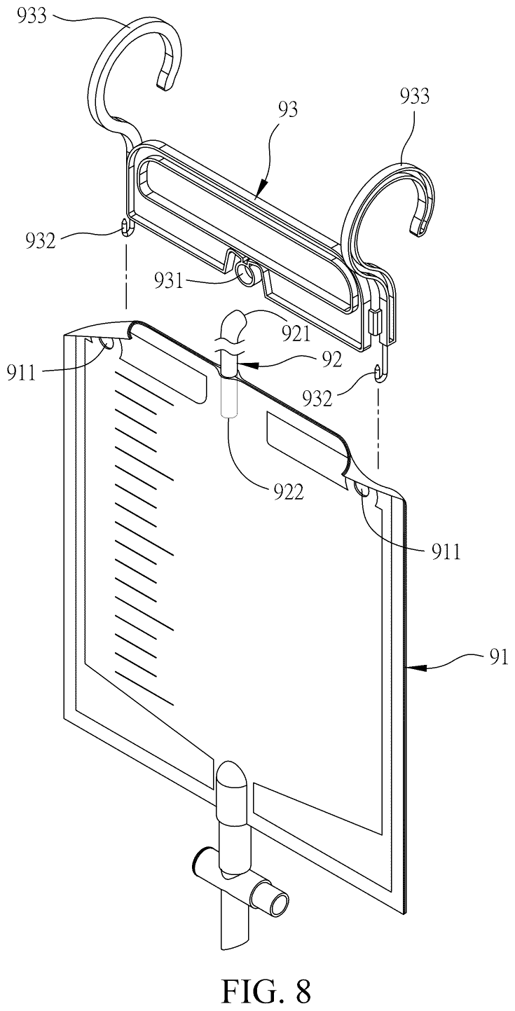

FIG. 9 is a schematic view illustrating the operation status of a conventional urinary bag.

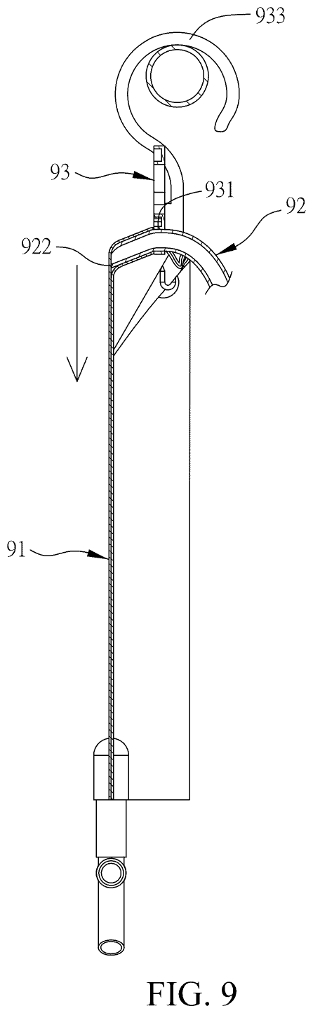

### DETAILED DESCRIPTION OF THE INVENTION

The aforementioned and further advantages and features of the present invention will be understood by reference to the description of the preferred embodiment in conjunction with the accompanying drawings where the components are illustrated based on a proportion for explanation but not subject to the actual component proportion.

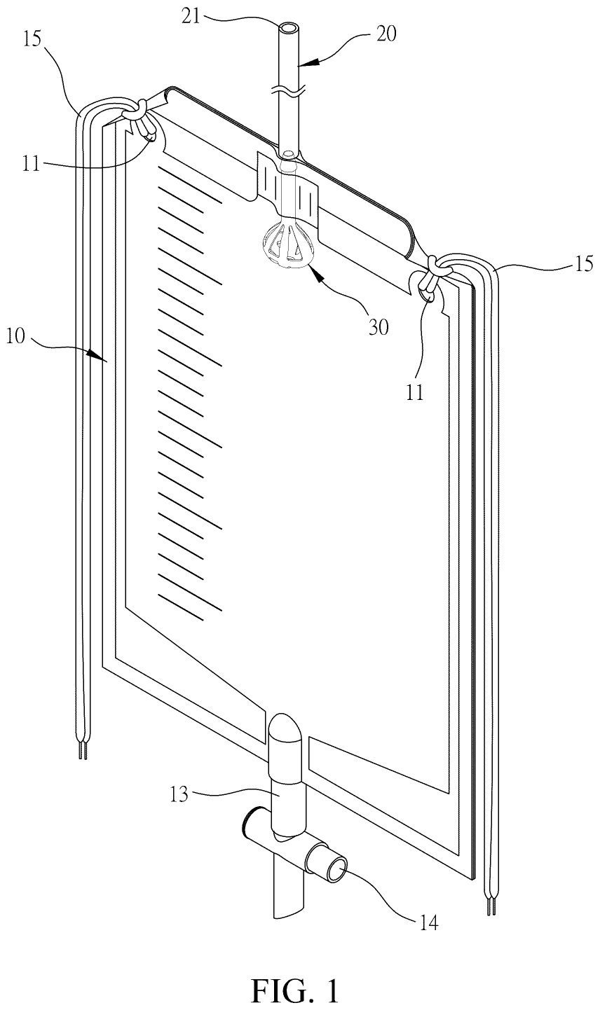

Referring to FIG. 1 to FIG. 4, the present invention provides an urinary bag reducing urine blockage comprises an urinary bag body 10, a catheter 20, and a bend preventing tube 30.

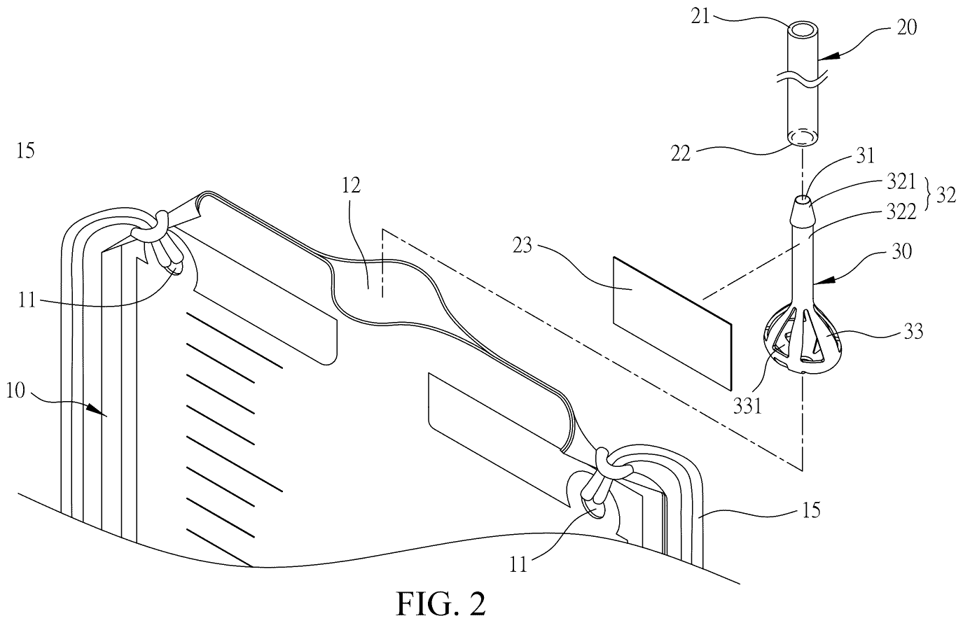

The urinary bag body 10 comprises two fixing bores 11 passing through two upper corners of the urinary bag body 10, and a through bore 12 on an upper middle side of the urinary bag body 10. The through bore 12 is arranged between the two fixing bores 11 (as shown in FIG. 1 and FIG. 2). The through bore 12 is in communication with the interior of the urinary bag body 10, and the urinary bag body 10 accommodates urine.

Also, an urine discharging tube 13 is connected to a lower middle side of the urinary bag body 10, with a valve device 14 disposed on the urine discharging tube 13, as shown in FIG. 1. Two positioning bands 15 are disposed in the two fixing bores 11, so that the urinary bag body 10 is allowed to be hung on the needed location by use of the two positioning bands 15.

The catheter 20 comprises a first connection part 21 and an opposite second connection part 22. The first connection part 21 is used to be connected with the urethra of the user (not shown), and the second connection part 22 is inserted into the urinary bag body 10 from the through bore 12 and placed inside the urinary bag body 10. Therein, the through bore 12 of the urinary bag body 10 is sealed through a processing method, which tightly fixes the catheter 20 in the through bore 12 of the urinary bag body 10. Therein, the processing method is allowed to be high-frequency welding or ultrasonic welding. In the embodiment, the processing method is high-frequency welding.

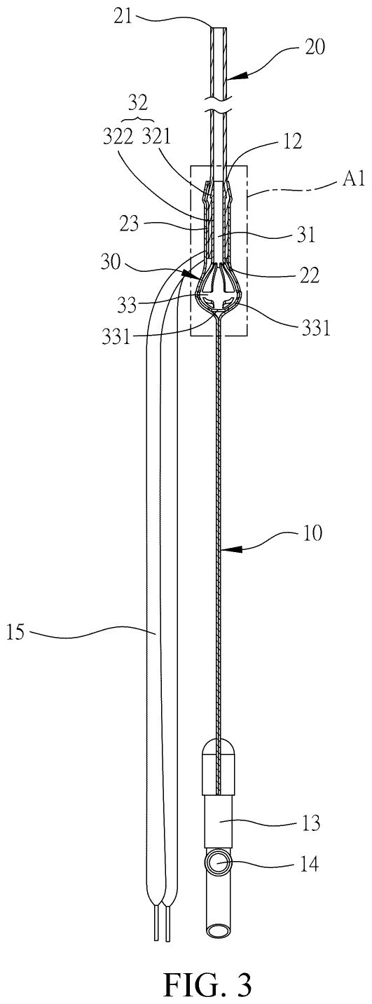

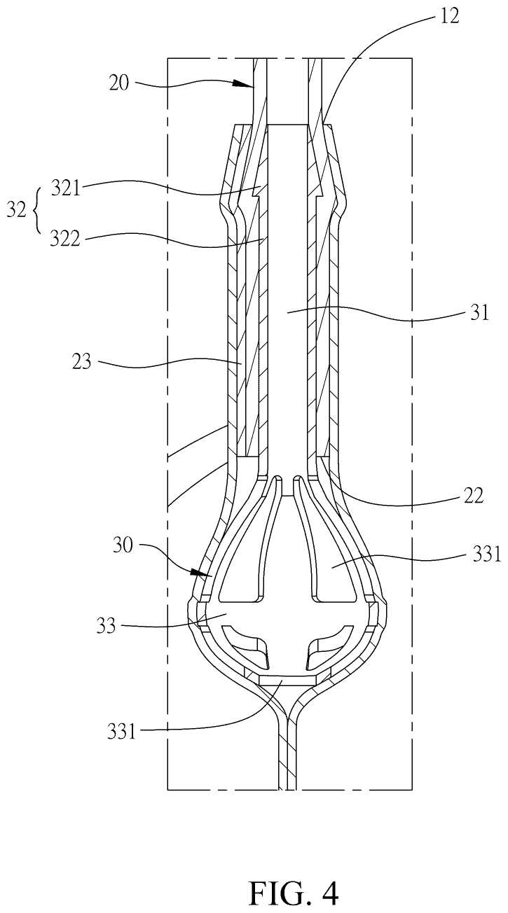

Also, a plastic sheet 23 is disposed between the second connection part 22 of the catheter 20 and the urinary bag body 10. The plastic sheet 23 wraps between the second connection part 22 of the catheter 20 and the urinary bag body 10 (as shown in FIG. 3 and FIG. 4). The plastic sheet 23 increases the tightness of the through bore 12 being sealed through the processing method, so as to achieve a tighter fixation of the urinary bag body 10 and the catheter 20, preventing the urine from refluxing or leaking from the second connecting portion 22.

The bend preventing tube 30 is connected between and in communication with the urinary bag body 10 and the catheter 20. The bend preventing tube 30 is integrally formed of a rigid plastic material, with a flow bore 31 passing therethrough. The bend preventing tube 30 comprises an insertion part 32 and an opposite supporting part 33. The second connection part 22 of the catheter 20 is mounted around the insertion part 32. The flow bore 31 is in communication with the catheter 20. The insertion part 32 keeps the catheter 20 to be straight. The supporting part 33 is placed inside the urinary bag body 10, as shown in FIG. 3 and FIG. 4.

Also, the insertion part 32 comprises a first portion 321 and a second portion 322. The first portion 321 is formed in a cone shape, and the second portion 322 is formed in a column shape. The largest outer diameter of the first portion 321 is larger than the outer diameter of the second portion 322; the length of the second portion 322 is larger than the length of the first portion 321, as shown in FIG. 3 and FIG. 4. Therein, when the catheter 20 is to be mounted around the insertion part 32 of the bend preventing tube 30, the second connection part 22 of the catheter 20 is efficiently mounted around the first portion 321 which has the smallest outer diameter. Then, with the variation from the expanding outer diameter of the first portion 321 to the smaller outer diameter of the second portion 322, the catheter 20 is stably mounted around the insertion part 32, achieving a detachment preventing effect.

Also, the outer diameter of the supporting part 33 is larger than the outer diameter of the first portion 321 of the insertion part 32. The supporting part 33 is able to support the interior of the urinary bag body 10. In the embodiment, the outer diameter of the supporting part 33 is larger than the largest diameter of the insertion part 32.

In addition, the supporting part 33 is formed in an ellipsoidal shape. A plurality of hollow holes 331 are distributed on the circumferential surface of the supporting part 33, and each hollow hole 331 is in communication with the flow bore 31. Therein, each hollow hole 331 is formed in a triangular shape, polygonal shape, or a combination thereof. In the embodiment, each hollow hole 331 is formed in the combination of triangular shape and polygonal shape. However, in the present invention, each hollow hole 331 is also allowed to be formed in other geometric shapes, not limited thereto.

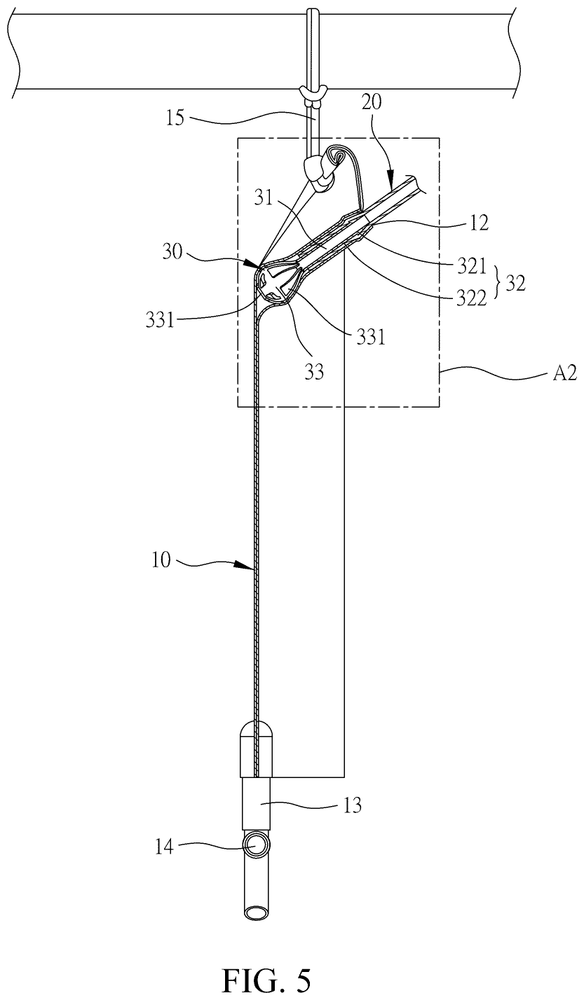

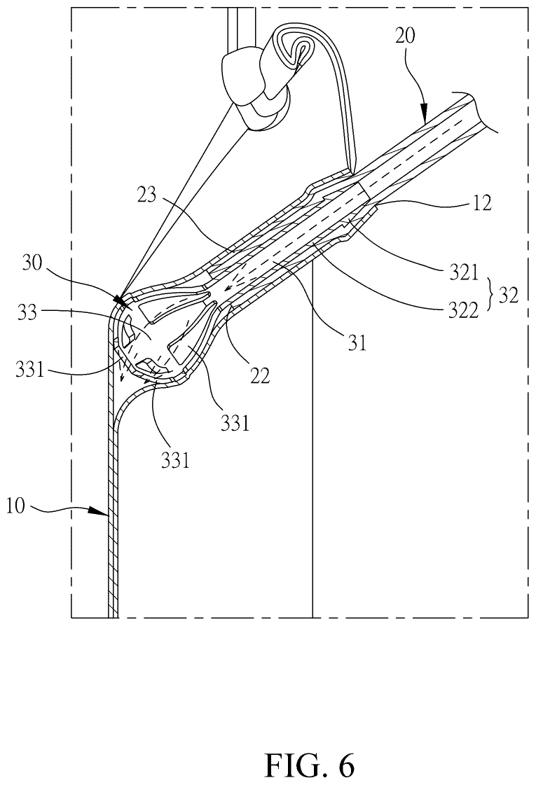

Referring to FIG. 5 and FIG. 6, during use, the two positioning bands 15 are fastened to the bedside or other suitable locations, and the urine of the user flows into the urinary bag body 10 through the catheter 20 to be collected. At the position where the catheter 20 normally bends in adjacent to the urinary bag body 10, the insertion part 32 keeps the catheter 20 to be straight, preventing the catheter 20 from being affected by the urinary bag body 10 to be bent. Furthermore, the supporting part 33 of the bend preventing tube 30 is pressed against the inner wall of the urinary bag body 10, so as to support the interior of the urinary bag body 10. Therefore, the urine flows from the catheter 20 through the flow bore 31 of the bend preventing tube 30 into the supporting part 33, and then smoothly enters the urinary bag body 10 through each hollow hole 331 of the supporting part 33, preventing the urine blockage from happening.

With the foregoing configuration, the present invention achieves following advantages.

In the urinary bag reducing urine blockage provided by the present invention, the bend preventing tube 30 is disposed on one end of the catheter 20 passing through the urinary bag body 10, and the supporting part 33 of the bend preventing tube 30 is placed inside the urinary bag body 10 to prevent the urine blockage, thereby ensuring the smooth urine drainage, improving the utility of the present invention.

The bend preventing tube 30 is integrally formed and inserted into the second connection part 22 of the catheter 20, achieving the structural simplicity, convenient assembly, and lower manufacturing cost, so as to meet the economic efficiency.

Each hollow hole 331 is formed in a non-circular shape, which allows the urine to be more smoothly inputted into the urinary bag body 10, so as to effectively prevent the urine blockage.

Although particular embodiments of the invention have been described in detail for purposes of illustration, various modifications and enhancements may be made without departing from the scope of the invention. Accordingly, the invention is not to be limited except as by the appended claims.

What is claimed is:

1. An urinary bag reducing urine blockage, comprising:
an urinary bag body having a through bore on an upper side thereof, the through bore being in communication with an interior of the urinary bag body;
a catheter connected and in communication with the urinary bag body, the catheter having a first connection part and an opposite second connection part, the first connection part used to be connected with an urethra of a user, the second connection part passing through the through bore of the urinary bag body and placed inside the urinary bag body; and
a bend preventing tube connected and in communication between the urinary bag body and the catheter, the bend preventing tube having an insertion part and a supporting part, with a flow bore passing through the insertion part and the supporting part, the second connection part of the catheter mounted around the insertion part, the flow bore in communication with the catheter, the insertion part keeping the catheter to be straight; the supporting part located inside the urinary bag body, an outer diameter of the supporting part being larger than an outer diameter of the insertion part, the supporting part supporting the interior of the urinary bag body.

2. The urinary bag reducing urine blockage of claim 1, wherein the supporting part is formed in an ellipsoidal shape; a plurality of hollow holes are distributed on a circumferential surface of the supporting part; each of the hollow holes is in communication with the flow bore.

3. The urinary bag reducing urine blockage of claim 1, wherein the through bore of the urinary bag body is sealed through a processing method; the processing method is applied to fix the catheter to the urinary bag body.

4. The urinary bag reducing urine blockage of claim 3, wherein the processing method is a high-frequency welding or ultrasonic welding.

5. The urinary bag reducing urine blockage of claim 1, wherein the urinary bag body comprises two fixing bores passing through the upper side of the urinary bag body; the through bore is arranged between the two fixing bores; two positioning bands are disposed in the two fixing bores.

6. The urinary bag reducing urine blockage of claim 1, wherein an urine discharging tube is connected to a lower middle side of the urinary bag body, and a valve device disposed on the urine discharging tube.

7. The urinary bag reducing urine blockage of claim 1, wherein a plastic sheet is disposed between the second connection part of the catheter and the urinary bag body.

8. The urinary bag reducing urine blockage of claim 1, wherein the bend preventing tube is integrally formed of a rigid plastic material.

9. The urinary bag reducing urine blockage of claim 1, wherein the insertion part comprises a first portion and a second portion; the first portion is formed in a cone shape, and the second portion is formed in a column shape; a largest outer diameter of the first portion is larger than an outer diameter of the second portion.

10. The urinary bag reducing urine blockage of claim 2, wherein each of the hollow holes is formed in a shape selected from a group consisting of triangular shape, polygonal shape, or a combination thereof.
